SEQUENCE LISTING (6 sequences)

SEQ ID NO: 1 - DNA, 111 bp, synthetic construct
  source 1..111: mol_type = other DNA
cacgatgaatttgagagacatgctgaagggacctttaccagtgatgtaagttcttatttggaaggccaagctgccaaggaattcattgcttggctggtgaaaggccgagga

SEQ ID NO: 2 - DNA, 29 bp, synthetic construct
  source 1..29: mol_type = other DNA
gggaatcacatcagtaactgagtgacttc

SEQ ID NO: 3 - DNA, 402 bp, synthetic construct
  source 1..402: mol_type = other DNA
atggcaaaattagagactgttactttaggtaacatcgggaaagatggaaaacaaactctggtcctcaatccgcgtggggtaaatcccactaacggcgttgcctcgctttcacaagcgggtgcagttcctgcgctggagaagcgtgttaccgtttcggtatctcagccttctcgcaatcgtaagaactacaaggtccaggttaagatccagaacccgaccgcttgcactgcaaacggttcttgtgacccatccgttactcgccaggcatatgctgacgtgaccttttcgttcacgcagtatagtaccgatgaggaacgagcttttgttcgtacagagcttgctgctctgctcgctagtcctctgctgatcgatgctattgatcagctgaacccagcgtattga

SEQ ID NO: 4 - DNA, 517 bp, synthetic construct
  source 1..517: mol_type = other DNA
ggcgaattaattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatg

SEQ ID NO: 5 - DNA, 21 bp, synthetic construct
  source 1..21: mol_type = other DNA
atggctcattaccgtaatgac

SEQ ID NO: 6 - DNA, 914 bp, synthetic construct
  source 1..914: mol_type = other DNA
taatacgactcactataggggaatcacatcagtaactgagtgacttcggcgaattaattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctcacctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccctggccaccacccatatgggatccatggccctgtggatgcgcctcctgcccctgctggcgctgctggccctctggggacctgacccagccgcagcccacgatgaatttgagagacatgctgaagggacctttaccagtgatgtaagttcttatttggaaggccaagctgccaaggaattcattgcttggctggtgaaaggccgaggagtcgacgcggccgcactcgagtgagatctgactgaaaaaaaaaaaaaaaaaaaaaaaaaaaaaagtttaaacactagtccgctgagcaataactagcataaccccttggggcctctaaacgggtcttgaggggttttttg

(19) United States
(12) Patent Application Publication
(10) Pub. No.: US 2023/0295640 A1
(43) Pub. Date: Sep. 21, 2023

(54) COMPOSITIONS AND METHODS FOR CONTROLLING BLOOD SUGAR LEVELS USING NANOCAPSULE-BASED DRUG DELIVERY SYSTEM

(71) Applicant: VL Biotech Inc., New Taipei (TW)

(72) Inventors: Hsin-Yi LIN, New Taipei (TW); Stanley KUNG, New Taipei (TW)

(21) Appl. No.: 18/184,965

(22) Filed: Mar. 16, 2023

Related U.S. Application Data
(60) Provisional application No. 63/320,819, filed on Mar. 17, 2022.

(51) Int. Cl. C12N 15/63; C07K 14/005; C07K 14/605; A61K 9/51; A61K 35/12; A61K 35/741; A61K 9/00; A61P 7/00

(52) U.S. Cl. CPC C12N 15/63; C07K 14/005; C07K 14/605; A61K 9/51; A61K 35/12; A61K 35/741; A61K 9/0053; A61P 7/00; C12N 2770/24023

(57) ABSTRACT

Disclosed herein are compositions and methods with enhanced capability to control blood sugar levels. The compositions are loaded in a nanocarrier for oral drug delivery. The composition comprises a first vector and a second vector. The first vector encodes one or more mRNAs. The one or more mRNAs encode a peptide with GLP-1 activity and are labeled with one or more capsid protein tags. The second vector encodes one or more capsid proteins. The one or more capsid proteins bind to the one or more capsid protein tags on the one or more mRNAs.

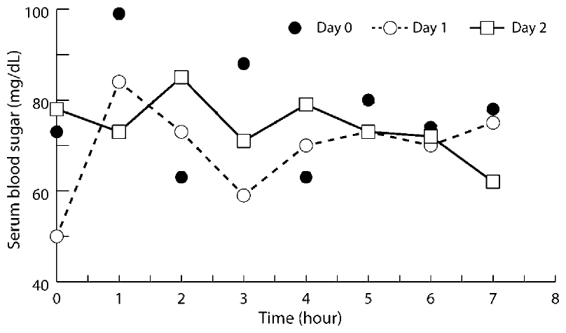

### REFERENCE TO AN ELECTRONIC SEQUENCE LISTING

The content of the electronic sequence listing (23HPUS0011_sequence listing.xml; Size: 8 KB; and Date of Creation: Mar. 16, 2023) is herein incorporated by reference in its entirety.

### FIELD OF THE INVENTION

The present invention relates to compositions and methods of use, in particular to therapeutic compositions with enhanced capability to control blood sugar levels in a human or animal subject, which are delivered by using nanocapsule-based drug delivery system. Compositions and methods of the present invention are particularly applicable to a range of disorders, including pre-diabetes, diabetes and related conditions.

### BACKGROUND OF THE INVENTION

Glucose in the blood is one of the main sources of energy. The stability of blood sugar levels is related to the normal operation of physiological functions in human or animal Once the blood sugar level is out of balance, it will lead to various diseases. The aberrant sugar level in the blood is commonly observed in many diseases. For example, diabetes mellitus or diabetes is a metabolic disorder characterized by a high blood sugar level. It has been shown that high blood sugar levels correlate with blood vessel damage, increasing the risk of heart attack, stroke, chronic kidney disease, and nerves, causing vision loss and cognition impairment. A person who has diabetes has a low insulin level or high insulin resistance.

In physiology, insulin is an endogenous peptide produced by the pancreas and plays an essential role in controlling blood sugar levels. Besides insulin, other endogenous peptides, such as glucagon-like peptide 1 (GLP-1), are associated with the blood sugar level. The discovered GLP-1 has been studied and used as the lead bioactive peptide for medicinal research in blood sugar level control and weight control. The synthesized GLP-1 derivatives have effectively controlled blood sugar level and weight by changing one's appetite. Thus, the GLP-1 derivatives are currently FDA-approved drugs. However, suppose one closely observes the serum level of these GLP-1 derivatives and their half-life. In that case, it discloses two issues: (i) the serum GLP-1 derivatives concentration is a thousand times higher than the native GLP-1, and (ii) the half-life of GLP-1 derivatives is over four thousand longer than the native, regardless of debates, are going on whether or not the above two issues will cause health problems, remaining more long-time usages and data to evaluate.

### SUMMARY OF THE INVENTION

The present disclosure provides a composition for controlling blood sugar levels. The composition comprises a first vector and a second vector. The first vector comprises the nucleotide sequence as denoted by SEQ ID NO: 1 and a capsid protein recognition sequence. The first vector encodes one or more mRNAs encoding a peptide with GLP-1 activity and the one or more mRNAs are labeled with one or more capsid protein tags. The second vector comprises one or more nucleotide sequences encoding one or more capsid proteins. The one or more capsid proteins are capable of self-assembling into one or more virus-like particles that encapsulate the one or more mRNAs such that the composition is fabricated as nanocapsule.

The present disclosure also provides a composition for controlling blood sugar levels. The composition comprises a first vector and a second vector. The first vector comprises the nucleotide sequence as denoted by SEQ ID NO: 6. The second vector comprises the nucleotide sequence as denoted by SEQ ID NO: 3. The nucleotide sequence of SEQ ID NO: 3 encodes one or more capsid proteins, and the one or more capsid proteins are capable of self-assembling into one or more virus-like particles that encapsulate one or more mRNAs encoded by the nucleotide sequence of SEQ ID NO: 6 such that the composition is fabricated as nanocapsule.

The present disclosure further provides a method for controlling blood sugar levels in a subject. The method includes administering a composition comprising one or more nanocapsules and/or encapsulated microbial cells to the subject. The one or more nanocapsules and/or encapsulated microbial cells are transformed by a first vector and a second vector. The first vector encodes one or more mRNAs. The one or more mRNAs encode a peptide with GLP-1 activity, and the one or more mRNAs are labeled with one or more capsid protein tags. The second vector comprises one or more nucleotide sequences encoding one or more capsid proteins. The one or more capsid proteins are capable of self-assembling into one or more virus-like particles that encapsulate the one or more mRNAs such that the composition is fabricated as the one or more nanocapsules or/and encapsulated microbial cells.

### BRIEF DESCRIPTION OF THE DRAWINGS

The FIGURE illustrates plots demonstrating blood sugar's homeostatic maintenance in the test hamster.

### DETAILED DESCRIPTION OF THE PREFERRED EMBODIMENTS

As used herein, the term “vector” used in reference to delivery of components refers to a nucleic acid capable of transporting between different genetic environments another nucleic acid to which it has been operatively linked. The term “express” in the context of the disclosure refers to transcription and/or translation of a specific nucleotide sequence driven by its promoter.

The disclosure relates to one or more nucleotide sequences encoding a peptide with GLP-1 activity, which particularly can be assembled into nanocapsule containing probiotics, for managing or controlling blood sugar levels. Thus, the one or more nucleotide sequences can be incorporated into pharmaceutical and/or therapeutic compositions suitable for oral administration.

The present disclosure discloses a first vector and a second vector, the first vector encodes one or more messenger ribonucleic acids (messenger RNAs, mRNAs) encoding a peptide with GLP-1 activity. The one or more mRNAs are further labeled with one or more capsid protein tags. The second vector encodes one or more capsid proteins (CPs), which may be self-assembled to form one or more hollow virus-like particles (virus-like particles, VLPs), then bind to and provide domain specificity to the one or more capsid protein tags on the one or more mRNAs, such that the one or more mRNAs are capable of being encapsulated in the one or more VLPs. Thus, the one or more mRNAs encoding the peptide with GLP-1 activity are carried in nanocarrier and will be delivered via nanocapsules after oral administration.

In some aspects, the pharmaceutical and/or therapeutic compositions comprise bio-products or compounds transcribed or translated from the first vector and the second vector described in the present disclosure.

The pharmaceutical and/or therapeutic composition may be used to prevent spikes in blood sugar levels of a human or animal subject. Therefore, in one aspect, it is contemplated that the compositions and methods of the disclosure are used to treat metabolic syndrome, pre-diabetes, diabetes and/or prevent the progress of diabetes.

In one aspect, the disclosure relates to one or more nanocapsule containing probiotics, which is assembled from one or more bio-products transcribed or translated from the first vector and the second vector. The bio-products may be either the mRNAs or the capsid proteins.

In another aspect, the disclosure relates to one or more nanocapsule assembled from one or more bio-products transcribed or translated from the first vector and the second vector. The bio-products may be either the mRNAs or the capsid proteins.

In yet another aspect, the disclosure relates to one or more VLPs assembled from one or more bio-products transcribed or translated from the first vector and the second vector. The bio-products may be either the mRNAs or the capsid proteins.

In a further aspect, the disclosure provides a use of the pharmaceutical and/or therapeutic composition of the disclosure in a preparation of a medicament for a treatment of metabolic syndrome, pre-diabetes, diabetes and/or prevent the progress of diabetes.

In another aspect, the disclosure provides a method for treating metabolic syndrome, pre-diabetes, diabetes and/or prevent the progress of diabetes, which comprises administering the pharmaceutical and/or therapeutic composition of the disclosure or the preparation thereof.

According to certain embodiments, the diabetes is selected from the group consisting of Type 1 diabetes, Type 2 diabetes and gestational diabetes.

In one embodiment, the first vector comprises the nucleotide sequence as denoted by SEQ ID NO: 1, and a capsid protein recognition sequence. The first vector is transcribed to form mRNA with the one or more capsid protein tags, which encodes a peptide with GLP-1 activity and can be used as a translation template in a cell of the human or animal subject to produce a peptide with GLP-1 activity in the human or animal subject's body, to endogenously produce GLP-1 in vivo. In addition, the one or more capsid protein tags can bind to a specific region of one or more capsid proteins because of a secondary structure formed by the capsid protein recognition sequence. In one embodiment, the capsid protein recognition sequence comprises the nucleotide sequence as denoted by SEQ ID NO: 2, and the one or more capsid protein tags formed by the capsid protein recognition sequence has a hairpin structure.

Further, the second vector comprises one or more nucleotide sequences encoding the one or more capsid proteins. In one embodiment, the one or more capsid proteins are shell proteins derived from bacteriophages, such as AP205 phage, Qbeta phage, MS2 phage, P22 phage and the like. In another embodiment, the second vector comprises the nucleotide sequence as denoted by SEQ ID NO: 3, and the one or more capsid proteins encoded by the nucleotide sequence of SEQ ID NO: 3 can be bound with the one or more capsid protein tags formed by the nucleotide sequence of SEQ ID NO: 2.

The first vector of the composition expresses the one or more mRNAs encoding the peptide with GLP-1 and the second vector of the composition expresses the one or more capsid proteins. The one or more capsid protein tags on the one or more mRNAs facilitate the combination of the capsid proteins and the one or more mRNAs. Consequently, the one or more capsid proteins are self-assembled into the one or more virus-like particles which can be utilized to deliver the one or more RNAs encoding the peptide with GLP-1 activity in the form of nanocapsules.

The first vector may further comprise an internal ribosome entry site (IRES) containing the nucleotide sequence as denoted by SEQ ID NO: 4. The internal ribosome entry site is placed between the nucleotide sequence of SEQ ID NO: 1 and the capsid protein recognition sequence. Moreover, the first vector may further comprise the nucleotide sequence as denoted by SEQ ID NO: 5, which encodes a secretion signal for promoting the synthesis of the peptide with GLP-1 activity. The first vector may include a promoter and a terminator for RNA polymerase to perform transcription, for example, a P11 promoter and a T 1 dh terminator may be used, but not limited to this.

In another embodiment, the composition of the disclosure comprises a first vector and a second vector. The first vector comprises the nucleotide sequence as denoted by SEQ ID NO: 6 encoding the one or more capsid protein tagged and one or more mRNAs which encodes a peptide with GLP-1 activity. The second vector comprises the nucleotide sequence as denoted by SEQ ID NO: 3 encoding one or more capsid proteins. The one or more capsid proteins encoded by the nucleotide sequence of SEQ ID NO: 3 can be combined with the one or more capsid protein tags formed by the nucleotide sequence of SEQ ID NO: 6.

The disclosure further provides a nanocapsule-based drug delivery system for delivering one or more mRNAs into the body of the human or animal subject to manage or control blood sugar level of the human or animal subject. In one embodiment, the nanocapsule-based drug delivery system is achieved by oral drug delivery vehicles such as encapsulated microbial cells. In one embodiment, the microbial cells may be probiotics. The microbial cell comprises a first vector and a second vector. The first vector encodes one or more mRNAs encoding a peptide with GLP-1 activity, and the one or more mRNAs are labeled with one or more capsid protein tags. The second vector encodes one or more capsid proteins, the one or more capsid proteins bind to the one or more capsid protein tags on the one or more mRNAs.

In one embodiment, the first vector and the second vector are transformed into a transformed cell, and the genetic information of the first vector and the second vector are expressed through the physiological mechanism of the transformed cell. In an example, the transformed cells are bacterial cells. In the process of the transformation (or the bacterial cells are produced), the first vector and the second vector are either transcribed into mRNAs or translated into capsid proteins; the bio-products are then self-assembled into the VLPs, encapsulated in the bacterial cells. As the VLPs are nano-scale particles, the one or more nucleotide sequences are assembled into nanocapsule containing bacterial cells. In an alternative example, the transformed cells are probiotics. In the process of the transformation (or the probiotics are produced), the first vector and the second vector are either transcribed into mRNAs or translated into capsid proteins; the bio-products are then self-assembled into the VLPs, encapsulated in the probiotics. As the VLPs are nano-scale particles, the one or more nucleotide sequences are assembled into nanocapsule containing probiotics.

Specifically, the first vector is expressed by the transformed cell to produce the one or more mRNAs with the one or more capsid protein tags, and the one or more mRNAs encode a peptide with GLP-1 activity in the body of the human or animal subject. The second vector is expressed by the transformed cell to form one or more capsid proteins. The products of the first vector and the second vector can be self-assembled into nanocapsule in the transformed cell by binding of the one or more capsid protein tags to the one or more capsid proteins.

Further, the oral drug delivery vehicles described above can be taken orally by patients, so that the nanocapsule can be transported into the organism through the carrying of the transformed cells, and released when they reach the intestinal tract. The released nanocapsules can be brought into the intestinal epithelial cells through endocytosis, and the one or more mRNAs in the released nanocapsules can be translated to form the peptides with GLP-1 activity through the translation mechanism of the epithelial cells. Thus, the composition of the disclosure can promote the body of the human or animal subject to produce the peptides with GLP-1 activity, which are more similar to endogenous GLP-1 than artificially synthesized GLP-1 derivatives, so as to be beneficial to stabilize blood sugar levels as well as weight control. Also, the burden and side effects of artificially synthesized GLP-1 on the human or animal's body will be decreased.

In one embodiment, the transformed cells are probiotics, which can be easily obtained and cultured from bacterial cultures. The probiotics may be Lactobacillus delbrueckii.

The following example is disclosed to demonstrate that the composition of the disclosure is able to endogenously produce GLP-1 in vivo, enabling the perpetuation of blood sugar homeostasis and body weight. The following examples are only for the purpose of illustration, and the scope of the disclosure is not limited by the examples. Those skilled in the art can utilize the disclosure and teachings of the disclosure to create other embodiments, aspects and variations without undue experimentation.

### Example 1

In the example, Lactobacillus delbrueckii is used as the vehicle for transformation of the first vector and the second vector. The first vector adopts pT7CFE1-NHA vector, which contains the nucleotide sequence of SEQ ID NO: 6. The second vector adopts pCDF-1b vector, which contains the nucleotide sequence of SEQ ID NO: 3. The first vector and the second vector are transformed into the Lactobacillus delbrueckii to form recombinant probiotics.

The test hamster fed with 50 mg of sugar water is used as a control group and the same test hamster fed with 50 mg of sugar water and the recombinant probiotics is used as an experiment group.

On Day 0, the test hamster is fed 50 mg of sugar water, and the serum blood sugar concentration of the test hamster is shown by solid circle as depicted in the FIGURE. On Day 1, the same test hamster is fed 50 mg of sugar water and also the recombinant probiotics, and the serum blood sugar concentration of the test hamster is shown by open circle as depicted in the FIGURE. On Day 2, the same test hamster is fed 50 mg of sugar water and also the recombinant probiotics again, and the serum blood sugar concentration of the test hamster is shown by open square as depicted in the FIGURE.

The trends of blood sugar concentration changes of the experiment group of Day 1 and the control group are similar. Compared with the control group, however, the peak of blood sugar concentration (at the first hour) of the experiment group of Day 1 is lowered by about 15%. In addition, the blood sugar concentration of the experiment group of Day 1 returns to normal range (i.e., the concentration before taking the sugar water) after four hours, which is shorter than the control group.

At the end of Day 1, the same test hamster is fed the sugar water and also the recombinant probiotics again. On Day 2, the peak of blood sugar concentration of the experiment group of Day 2 is almost the same as which of Day 1. However, drastic change in blood sugar concentration is inhibited, which means the blood sugar homeostasis is preserved.

The probiotics can deliver and release the nano-capsules as described above in the gastrointestinal tract, where the particles get into the epithelium cells via endocytosis. The endocytic nano-capsules then open up and release the contents (the nucleotide sequence carried in the first vector), for template-codon translation using the host's translation machinery. Once the GLP-1 has been made in the cell, the cytosolic GLP-1 will be exported into circulation.

What is claimed is:

1. A composition for controlling blood sugar level, comprising:
a first vector, comprising the nucleotide sequence as denoted by SEQ ID NO: 1 and a capsid protein recognition sequence, wherein the first vector encodes one or more mRNAs encoding a peptide with GLP-1 activity and the one or more mRNAs are labeled with one or more capsid protein tags; and
a second vector, comprising one or more nucleotide sequences encoding one or more capsid proteins, wherein the one or more capsid proteins are capable of self-assembling into one or more virus-like particles that encapsulate the one or more mRNAs such that the composition is fabricated as nanocapsule.

2. The composition according to claim 1, wherein the capsid protein recognition sequence in the first vector comprises the nucleotide sequence as denoted by SEQ ID NO: 2, and the second vector comprises the nucleotide sequence as denoted by SEQ ID NO: 3.

3. The composition according to claim 1, wherein the first vector further comprises an internal ribosome entry site (IRES) between the nucleotide sequence of SEQ ID NO: 1 and the capsid protein recognition sequence.

4. The composition according to claim 1, wherein the first vector comprises the nucleotide sequence as denoted by SEQ ID NO: 5.

5. A composition for controlling blood sugar levels, comprising:
a first vector, comprising the nucleotide sequence as denoted by SEQ ID NO: 6; and
a second vector, comprising the nucleotide sequence as denoted by SEQ ID NO: 3;
wherein the nucleotide sequence of SEQ ID NO: 3 encodes one or more capsid proteins, and the one or more capsid proteins are capable of self-assembling into one or more virus-like particles that encapsulate one or more mRNAs encoded by the nucleotide sequence of SEQ ID NO: 6 such that the composition is fabricated as nanocapsule.

6. A method for controlling blood sugar level in a subject, comprising:
administering a composition comprising one or more nanocapsules and/or encapsulated microbial cells to the subject, wherein the one or more nanocapsules and/or encapsulated microbial cells are transformed by a first vector and a second vector, the first vector encoding one or more mRNAs, wherein the one or more mRNAs encode a peptide with GLP-1 activity, and the one or more mRNAs are labeled with one or more capsid protein tags, the second vector comprising one or more nucleotide sequences encoding one or more capsid proteins, wherein the one or more capsid proteins are capable of self-assembling into one or more virus-like particles that encapsulate the one or more mRNAs such that the composition is fabricated as the one or more nanocapsules and/or encapsulated microbial cells.

7. The method according to claim 6, wherein the first vector comprises the nucleotide sequence as denoted by SEQ ID NO: 1 and a capsid protein recognition sequence.

8. The method according to claim 6, wherein the capsid protein recognition sequence in the first vector comprises the nucleotide sequence as denoted by SEQ ID NO: 2, and the second vector comprises the nucleotide sequence as denoted by SEQ ID NO: 3.

9. The method according to claim 6, wherein the first vector further comprises an internal ribosome entry site (IRES) between the nucleotide sequence of SEQ ID NO: 1 and the capsid protein recognition sequence.

10. The method according to claim 6, wherein the first vector comprises the nucleotide sequence as denoted by SEQ ID NO: 5.

11. The method according to claim 6, wherein the first vector comprises the nucleotide sequence as denoted by SEQ ID NO: 6, and the second vector comprises the nucleotide sequence as denoted by SEQ ID NO: 3.

12. The method according to claim 6, wherein the encapsulated microbial cell is in the form of probiotic.

13. The method according to claim 12, wherein the probiotic is Lactobacillus delbrueckii.

14. The method according to claim 6, wherein the composition is orally administered to the subject.
